(19) United States
(12) Patent Application Publication
(10) Pub. No.: US 2025/0120998 A1
(43) Pub. Date: Apr. 17, 2025

(54) REPAIR OF ENDOTHELIAL GLYCOCALYX

(71) Applicant: United Therapeutics Corporation, Silver Spring, MD (US)

(72) Inventors: Sarah Hogan, Raleigh, NC (US); Kelly Guthrie, Raleigh, NC (US); Ryan Bonvillain, Cary, NC (US); Chris Givens, Chapel Hill, NC (US); Thomas Petersen, Durham, NC (US)

(73) Assignee: United Therapeutics Corporation, Silver Spring, MD (US)

(21) Appl. No.: 18/917,505

(22) Filed: Oct. 16, 2024

Related U.S. Application Data
(60) Provisional application No. 63/590,949, filed on Oct. 17, 2023.

(51) Int. Cl. A61K 31/728; A61K 31/198; A61K 31/737; A61P 11/00

(52) U.S. Cl. CPC A61K 31/728; A61K 31/198; A61K 31/737; A61P 11/00

(57) ABSTRACT

Compositions and methods relating to glycosaminoglycan components are disclosed. For example, a composition including glycosaminoglycan components are administered to organs before, during, or after ex vivo organ perfusion to improve organ transplantation. The improvements include reduced MMP levels, reduced apoptotic markers, reduced endothelial activation, and increased organ function. Such methods and compositions are useful for organ and tissue transplantation and storage or shipment of harvested organs and tissues.

### CROSS REFERENCE TO RELATED APPLICATION

This application claims the benefit under 35 U.S.C. § 119 (e) of U.S. Provisional Application No. 63/590,949, filed Oct. 17, 2023, the contents of which are incorporated herein by reference in their entirety.

### FIELD OF THE INVENTION

This disclosure relates to the use of glycosaminoglycan components for improving the method of transplants for tissues and organs, such as lungs, and to the therapeutic use of glycosaminoglycan components.

### BACKGROUND OF THE INVENTION

The following description of the background of the present technology is provided simply as an aid in understanding the present technology and is not admitted to describe or constitute prior art to the present technology.

The endothelial glycocalyx (EG) is layer of proteoglycans and glycosaminoglycans that lines the luminal surface of the endothelial cells in blood vessels. The EG layer plays a role as a barrier to circulating cells and large molecules and to regulate transvascular exchange of water and other solutes. EG damage and disruption may be triggered by ischemia and reperfusion. Dysfunction of the EG can occur through partial or complete loss of its components and cells and result in vascular permeability. EG shedding is driven by enzymes, such as the matrix metalloproteinases (MMPs).

Organ transplantation is a necessary life-saving therapy for patients suffering from various end-stage diseases. One such example is lung transplantation. Ex vivo lung perfusion (EVLP) is a system by which a pair of lungs is preserved outside of a body, allowing the lungs to be evaluated and held for a limited period of time before transplant. Successful transplantation is limited by primary graft dysfunction (PGD), which is a major cause of early morbidity and mortality. Severe PGD is characterized as the ratio of partial pressure of oxygen in the arterial blood (PaO₂) and fraction of inspired oxygen (FiO₂) less than 200 mmHg with the presence of pulmonary infiltrates, such as accumulation of lung water.

There is a continuing need in the field of transplantation for methods of treatment and compositions that are capable of improving function and viability of tissues and organs. Because of the associations between negative outcomes after organ transplantation and EG dysfunction, improving EG integrity could promote viability and function of tissues and organs following transplantation.

### SUMMARY OF THE INVENTION

In one aspect, the present disclosure relates to a composition of glycosaminoglycan components and the use of such a composition for improving tissue and organ function. As shown herein, administration of a composition of glycosaminoglycan components can improve organ function.

In another aspect, the present disclosure provides a composition for treating an organ to improve organ transplantation, the composition comprising an effective amount of at least one glycosaminoglycan component. In some embodiments according to this first aspect, the at least one glycosaminoglycan component comprises high molecular weight hyaluronic acid, sulodexide, or a combination of high molecular weight hyaluronic acid and sulodexide. In some embodiments of this first aspect, the composition further comprises at least one additional component selected from the group consisting of: isolated mitochondria, total parenteral nutrition (TPN), an antioxidant, and a thrombolytic agent. In some embodiments of this first aspect, the antioxidant is N-acetyl cysteine. In some embodiments of this first aspect, the thrombolytic agent is human tissue type plasminogen activator. In some embodiments of this first aspect, high molecular weight hyaluronic acid is administered in a dose of 0.01 mg to 15 mg. In some further embodiments of this first aspect, an initial dose of high molecular weight hyaluronic acid is administered at hour 1 of ex vivo organ perfusion and optionally additional doses administered every 6 hours following the initial dose. In some embodiments of this first aspect, sulodexide is administered in a dose of 0.067 mg/kg to 30 mg/kg. In some further embodiments of this first aspect, an initial dose of sulodexide is administered at hour 2 and optionally additional doses administered hourly following the initial dose. In some embodiments of this first aspect, the composition further comprises an antioxidant and/or a thrombolytic agent. In some further embodiments of this first aspect, the antioxidant is N-acetyl cysteine. In some further embodiments of this first aspect, the thrombolytic agent is human tissue type plasminogen activator. In some embodiments of this first aspect, the organ is a lung, a liver, or a kidney.

In a second aspect, the present disclosure provides a method of improving organ transplantation, comprising administration of a composition according to the first aspect to an organ before or during transplantation. In some embodiments of the second aspect, the glycosaminoglycan components comprise comprises high molecular weight hyaluronic acid, sulodexide, or a combination of high molecular weight hyaluronic acid and sulodexide. In some embodiments of this second aspect, the composition further comprises at least one additional component selected from the group consisting of: isolated mitochondria, total parenteral nutrition (TPN), an antioxidant, and a thrombolytic agent. In some embodiments of this second aspect, the antioxidant is N-acetyl cysteine. In some embodiments of this second aspect, the thrombolytic agent is human tissue type plasminogen activator. In some embodiments of the second aspect, high molecular weight hyaluronic acid is administered in a dose of 0.01 mg to 15 mg. In some embodiments of the second aspect, sulodexide is administered in a dose of 0.067 mg/kg to 30 mg/kg. In some further embodiments of this second aspect, the administration of the composition comprises perfusing the organ with the composition. In some embodiments of this second aspect, the organ is perfused with the composition for at least one hour. In some other embodiments of this second aspect, the administration of the composition comprises administering one or more doses of the composition to the organ before transplantation. In some embodiments of this second aspect, the organ exhibits a decrease in: a) circulating MMPs, b) markers of apoptosis, and/or c) endothelial cell activation, as compared to an organ perfused with a solution lacking glycosaminoglycan components. In some embodiments of this second aspect, the composition further comprises an antioxidant and/or a thrombolytic agent. In some embodiments according to this second aspect, the organ is a lung, a liver, or a kidney. In some embodiments according to this second aspect, the organ is a bioengineered organ.

Further objects and advantages of the present invention will be clear from the description that follows.

### BRIEF DESCRIPTION OF THE DRAWINGS

FIG. 1 (FIG. 1) shows an exemplary ex vivo lung perfusion (EVLP) set up used in the Examples.

FIGS. 2A-21 (FIGS. 2A-21) show the results of the experiments evaluating the functional outcomes of lungs undergoing EVLP. FIG. 2A shows measurement of dynamic compliance for different lungs subjected to EVLP plotted against the observed time that the lung was able to withstand EVLP. The line illustrates the correlation between dynamic compliance and time to failure based on the samples. FIG. 2B shows the measurement of Steen loss in the first two hours (hollow circles) and of average Steen loss over the course of EVLP (solid circles) for individual lungs. The top line on FIG. 2B depicts the correlation between Steen loss after the first two hours of EVLP and time to failure based on the samples tested, while the bottom line depicts the correlation between Steen loss over the course of EVLP and time to failure. FIG. 2C shows the partial pressure of oxygen in arterial blood to the fraction of inspired oxygen (pO₂/FiO₂) measured at take-down plotted against the observed EVLP duration for each lung tested, with the line depicting the correlation between pO₂/FiO₂ and time to failure. FIG. 2D shows the pulmonary vascular resistance (PVR) measured at take-down plotted for each lung against the observed EVLP duration, with the correlation between PVR and time to failure represented by the line. FIG. 2E shows dynamic compliance at take-down (solid squares) and the slope of dynamic compliance during EVLP (solid triangles) plotted against average Steen loss per hour over the course of EVLP. FIG. 2F shows dynamic compliance at take-down plotted against rate of accumulation of circulating cytochrome C. FIG. 2G depicts dynamic compliance at take-down plotted against the rate of accumulation of circulating syndecan-1. FIG. 2H shows dynamic compliance at take-down plotted against the levels or rate of accumulation of VCAM1 (solid circles) and the rate of accumulation of EpCAM (solid squares).

FIGS. 3A-3D (FIGS. 3A-3D) depicts the levels of proteins collected from bronchoalveolar lavage (BAL) at hour one of EVLP plotted against various measurements of glycocalyx dysfunction and reduced lung function. FIG. 3A shows the level of MMP detected in the BAL (circles) and the ratio of MMP to TIMP detected in the BAL (squares) plotted against average Steen loss per hour. FIG. 3B shows the level of MMP detected in the BAL (circles) and the ratio of MMP to TIMP detected in the BAL (squares) plotted against average dynamic compliance over the course of EVLP. FIG. 3C shows the level of MMP detected in the BAL (circles) and the ratio of MMP to TIMP detected in the BAL (squares) plotted against dynamic compliance at take-down. FIG. 3D shows the level of EpCAM detected at hour 1 of EVLP plotted against dynamic compliance at take-down.

FIGS. 4A-4N (FIGS. 4A-4N) depicts the results of experiments measuring glycocalyx dysfunction and lung function over the course of EVLP for lungs treated with glycosaminoglycan components and control lungs that were not treated. FIG. 4A shows the measurement of dynamic compliance plotted against EVLP duration. Data for the control lungs that have not been treated with the composition comprising glycosaminoglycans are shown in hollow circles while the data for the lungs that have been treated with the composition comprising glycosaminoglycan are shown in solid squares. FIG. 4B shows the measurement of static compliance plotted against EVLP duration. Data for the control lungs that have not been treated with the composition comprising glycosaminoglycans are shown in hollow circles while the data for the lungs that have been treated with the composition comprising glycosaminoglycan are shown in solid squares. FIG. 4C shows the measurement of the partial pressure of oxygen in arterial blood over the fraction of inspired oxygen (pO₂/FiO₂) over the course of EVLP. Data for the control lungs that have not been treated with the composition comprising glycosaminoglycans is shown in hollow circles. Data for the lungs that have been treated with the composition comprising glycosaminoglycan is shown in solid squares. FIG. 4D shows the measurement of the pulmonary vascular resistance over the course of EVLP. The data for the control lungs that have not been treated with the composition comprising glycosaminoglycans is shown in the hollow circles. The data for the lungs that have been treated with the composition comprising glycosaminoglycans is shown in the solid squares. FIG. 4E shows the consumption of glucose over the duration of EVLP for control lungs and lungs treated with the composition comprising glycosaminoglycans. The data for the control lungs that have not been treated with the composition comprising glycosaminoglycans is shown in the hollow circles. The data for the lungs that have been treated with the composition comprising glycosaminoglycans is shown in the solid squares. FIG. 4F shows the production of lactate by control lungs and lungs treated with the composition comprising glycosaminoglycans. The data for the control lungs that have not been treated with the composition comprising glycosaminoglycans is shown in the hollow circles. The data for the lungs that have been treated with the composition comprising glycosaminoglycans is shown in the solid squares. FIG. 4G shows the measurement of Steen consumption over the course of EVLP. The data for the control lungs that have not been treated with the composition comprising glycosaminoglycans is shown in hollow circles. Data for the lungs that have been treated with the composition comprising glycosaminoglycan is shown in solid squares. FIG. 4H show staining for hyaluronic acid (HA) at hour 4 of EVLP, hour 24 of EVLP without treatment with the composition comprising glycosaminoglycans, and hour 24 of EVLP with treatment with the composition comprising glycosaminoglycans. FIG. 4I shows the slope or rate of accumulation of VCAM1 levels measured for control lungs (grey squares) and lungs treated with the composition comprising glycosaminoglycans (black triangles) plotted against dynamic compliance at take-down. FIG. 4J shows the average rate of accumulation of VCAM1 to dynamic compliance slope for the control lungs and the treated lungs. FIG. 4K shows syndecan-1 levels measured for control lungs (solid circles) and lungs treated with the composition comprising glycosaminoglycans (hollow circles) plotted against dynamic compliance at take-down. FIG. 4L shows the average rate of accumulation of syndecan-1 to dynamic compliance slope for the control lungs and the treated lungs. FIG. 4M shows MMP levels measured at hour 1 of EVLP for control lungs (black circles) and lungs treated with the composition comprising glycosaminoglycans (grey circles) plotted against dynamic compliance at take-down. FIG. 4N shows EpCAM levels measured for control lungs (solid squares) and lungs treated with the composition comprising glycosaminoglycans (solid triangles) plotted against dynamic compliance at take-down.

FIGS. 5A-5C (FIGS. 5A-5C) shows MMP levels and MMP/TIMP measured in the bronchoalveolar lavage (BAL) of each lung at hour 1 of EVLP plotted against various measurements of lung function, with the untreated controls shown in solid squares for MMP/TIMP and solid circles for total MMP levels and the treated samples shown in hollow squares for MMP/TIMP and hollow circles for total MMP levels. FIG. 5A shows MMP levels and MMP/TIMP measured in the BAL of each lung at hour 1 of EVLP plotted against Steen loss per hour. FIG. 5B depicts MMP levels and MMP/TIMP measured in the bronchoalveolar lavage (BAL) of each lung at hour 1 of EVLP plotted against the average dynamic compliance over the duration of EVLP . . . FIG. 5C depicts MMP levels and MMP/TIMP measured in the bronchoalveolar lavage (BAL) of each lung at hour 1 of EVLP plotted against the dynamic compliance at take-down. MMP levels are shown in circles, with solid circles representing the data from untreated lungs and hollow circles representing the data from the treated lungs. MMP/TIMP ratios are shown in squares, with solid squares representing the data from the untreated lungs and hollow squares representing the data from the treated lungs.

FIG. 6 (FIG. 6) depicts the slope of cytochrome C accumulation over the course of EVLP plotted against dynamic compliance at take-down. Data from the untreated samples are shown in solid circles. Data from the treated samples are shown in hollow circles.

FIG. 7 (FIG. 7) depicts the volcano plot of the differentially expressed proteins in lungs following treatment with the composition comprising glycosaminoglycans. The grey line represents the cut-off for proteins considered differentially expressed.

### DETAILED DESCRIPTION OF THE DRAWINGS

Disclosed herein is a composition to improve organ transplantation, comprising at least one glycosaminoglycan component. A glycosaminoglycan (GAG) is a long, linear polysaccharide comprised of repeating disaccharide units with pleiotropic biological functions, including but not limited to hyaluronic acid, sulodexide, sulfated GAGs such as dermatan sulfate, chondroitin sulfate, heparan sulfate, keratan sulfate, and to a lesser extent heparin. Glycocalyx integrity is related to organ quality for transplantation and can be measured by detecting soluble endothelial cell (EC) activation proteins.

For example, for lung transplantation, soluble EC activation proteins detected during EVLP are associated with organ rejection pre-transplant or PGD post-transplant. In addition, circulating soluble adhesion molecules shed from the vascular endothelium, which are another sign of endothelial activation, are negatively correlated with EVLP functional metrics, such as dynamic compliance, a measure of the lung's elasticity and health.

Taken together, these findings suggest that loss of glycocalyx integrity may lead to worse transplant outcomes. Thus, supporting glycocalyx integrity may present a novel avenue of promoting successful organ transplantation.

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein for the purpose of describing particular embodiments only and is not intended to be limiting. The present disclosure shows that administration of a composition of glycosaminoglycan components can reduce markers of endothelial damage and activation while increasing lung function. The specific structural and functional details disclosed herein are not to the interpreted as limiting but merely as a basis for the claims and as a representative basis for teaching one skilled in the art to variously employ the embodiments described herein.

### I. Definitions

To facilitate an understanding of the present invention, a number of terms and phrases are defined below. Unless defined otherwise, all technical and scientific terms used herein generally have the same meaning as commonly understood by one of ordinary skill in the art to which this technology belongs.

As used in this specification and the appended claims, the singular forms “a,” “an,” and “the” include plural referents unless the context clearly dictates otherwise. For example, reference to a “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” or “approximately” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

“Administering” (or any form of administration such as “administered”) means delivery of an effective amount of composition to a subject as described herein. Exemplary routes of administration include, but are not limited to, injection (such as subcutaneous, intramuscular, intradermal, and intravenous), oral, dermal, and transdermal routes.

The term “ex vivo” refers to a condition applied to a cell, tissue, or other sample obtained from an organism that takes place outside the organism.

The terms “hypoxia,” “hypoxic,” and “hypoxic conditions” refer to a condition under which an organ, tissue, or cell receive an inadequate supply of oxygen.

The term “ischemia” is defined as an insufficient supply of blood to a specific organ, tissue, or cell. A consequence of decreased blood supply is an inadequate supply of oxygen to the organ, tissue, or cell (hypoxia). Prolonged hypoxia may result in injury to the affected organ, tissue, or cell.

As used herein, the term “organ” refers to a part or structure of a body, which is adapted for a special function or functions. In a particular embodiment, the organ is the lungs.

The term “reperfusion” refers to the resumption of blood flow in a tissue or organ following a period of ischemia.

The term “sample” is used in its broadest sense.

As used herein, the term “subject” includes any human or nonhuman animal. The term “nonhuman animal” includes, but is not limited to, vertebrates such as nonhuman primates, sheep, dogs, cats, rabbits, ferrets, rodents (such as mice, rats and guinea pigs), avian species (such as chickens), amphibians, and reptiles. In preferred embodiments, the subject is a mammal such as a nonhuman primate, sheep, dog, car, rabbit, ferret, or rodent. In more preferred embodiments, the subject is a human.

The terms “comprising,” “including,” “having” and the like, as used with respect to embodiments, are synonymous. It is understood that wherever embodiments described herein with the language “comprising,” otherwise analogous embodiments described in terms of “consisting of” and/or “consisting essentially of” are also provided.

For the purpose of the description, a phrase in the form “A/B” or in the form “A and/or B” means (A), (B) or (A and B). For the purposes of the description, a phrase in the form “at least one of A, B, and C” means (A), (B), (C), (A and B), (A and C), (B and C) or (A, B and C).

The term “Steen” as used herein refers to a solution for preserving an organ, preferably comprising serum albumin (preferably at a concentration of 2-105 g/L), a scavenger and coating compound, preferably dextran compounds and derivatives thereof having essentially the same structure (preferably at a concentration of 1-55 g/L weight), and a physiological serum concentration of salts and nutrients in a physiologically acceptable medium. One suitable Steen solution comprises Dextran 40 at a concentration of 5 g/L, sodium chloride, dextrose monohydrate, potassium chloride, calcium chloride dihydrate, sodium dihydrogen, phosphate dihydrate, sodium bicarbonate, magnesium chloride, hexahydrate, and human serum albumin, 25% (70 g/L). Suitable Steen solutions are also provided in U.S. Pat. No. 7,255,983, the entire contents of which are incorporated by reference herein.

The description may use the terms “embodiment” or “embodiments,” which may refer to one or more of the same or different embodiments.

### II. Composition to Improve Organ Transplantation

Disclosed herein is a composition to treating an organ before or during ex vivo organ perfusion to improve organ transplantation, including at least one glycosaminoglycan component. In some embodiments, the composition is administered during ex vivo organ perfusion. In some embodiments, the composition contains at least one glycosaminoglycan component that includes but is not limited to high molecular weight hyaluronic acid, sulodexide, or both high molecular weight hyaluronic acid and sulodexide. High molecular weight hyaluronic acid was selected as it may be capable of inhibiting leakiness and restoring the glycocalyx.

In some embodiments, the organ treated with the composition is, but is not limited to, a lung, a liver, or a kidney. In some embodiments, the organ is a xenotransplant organ. In some embodiments, the lung is a human lung. In some embodiments, a liver is a human liver. In some embodiments, the kidney is a human kidney. In some embodiments, the organ treated with the composition is, but is not limited to, a bioengineered organ. The bioengineered organ may be a mechanical bioengineered organ, a biomechanical bioengineered organ, or a biological or bioartificial bioengineered organ. In some further embodiments, the bioengineered organ is, but is not limited to, a bioengineered lung, a bioengineered liver, or a bioengineered kidney.

In some embodiments, the composition is administered to the organ before, during, or after transplantation. In some embodiments, the composition is administered to the organ before or during ex vivo organ perfusion. In some embodiments, the composition is administered at the time of or after organ procurement. In some embodiments, the composition is administered before shipment. In some embodiments, the composition is administered after shipment. In some embodiments, the composition is administered before ex vivo organ perfusion begins. In some embodiments, the composition is administered during ex vivo organ perfusion. In some embodiments, the composition is administered after ex vivo organ perfusion ends. In some embodiments, the composition is administered before transplantation occurs. In some embodiments, the composition is administered during transplantation. In some embodiments, the composition is administered after transplantation is completed. In some embodiments, the composition is administered to the organ via, but not limited to, perfusion or injection. In some embodiments, the composition is administered via perfusion for at least 1, 2, 3, 4, 5, 6, 7, 8, 9, 10, 11, 12, or more hours. In some embodiments, the composition is administered in doses over the course of perfusion. In some embodiments, the composition is administered to the organ via delivery to the airway. In embodiments wherein the organ is a bioengineered organ, the composition may be administrated before cell seeding or after cell seeding or at any of the times specified above.

In some embodiments, high molecular weight hyaluronic acid is administered in a dose of about 0.01 mg to about 15 mg. In some embodiments, an initial dose of high molecular weight hyaluronic acid is administered at hour 1 of ex vivo organ perfusion and optionally additional doses are administered every 6 hours following the initial dose. In some embodiments, sulodexide is administered in a dose of about 0.067 mg/kg to about 30 mg/kg. In some embodiments, an initial dose of sulodexide is administered at hour 1 of ex vivo organ perfusion and optionally additional hourly following the initial dose. In some embodiments, an initial dose of sulodexide is administered at hour 2 of ex vivo organ perfusion and optionally additional doses administered hourly following the initial dose. In some embodiments, the initial dose of high molecular weight hyaluronic acid and/or sulodexide are the same dose as the optional additional doses. In some embodiments, the initial does of high molecular weight hyaluronic acid and/or sulodexide are different from the optional additional doses. In some embodiments, the composition is introduced to the solution for ex vivo organ perfusion including but not limited to dextran 40, sodium chloride, dextrose monohydrate, potassium chloride, calcium chloride dihydrate, sodium dihydrogen, phosphate dihydrate, sodium bicarbonate, magnesium chloride, hexahydrate, and human serum albumin (25%). In some embodiments the solution for ex vivo organ perfusion is, but is not limited to, Steen solution.

In some embodiments, the composition may include an antioxidant. Oxidative stress causes further disruption of the glycocalyx during ischemia-reperfusion, which leads to secondary inflammatory responses. Providing an antioxidant may reduce reactive oxygen species (ROS) and tissue edema during ex vivo organ perfusion. The included antioxidant may be but is not limited to N-acetyl cysteine.

In some embodiments, the composition may also include a thrombolytic agent. Thrombolytic agents dissolve blood clots, improve blood flow, and prevent damage to organs. The included thrombolytic agent may be but is not limited to human tissue type plasminogen activator.

In some embodiments, the composition may also include isolated mitochondria. In some further embodiments, the isolated mitochondria may be fresh mitochondria or frozen mitochondria. Isolated mitochondria may be used to improve the metabolism of the organ during EVLP.

In some embodiments, the composition may also include total parenteral nutrition (TPN). TPN may also be used to improve metabolism of the organ during EVLP.

In preferred embodiments, administration of the composition results in increased organ function. Examples of the improved organ function may be, but are not limited to, indicators associate with a reduction in edema or vascular leak.

Some non-limiting examples of improved organ function after administration of the composition include reduced circulating MMP, reduced markers of apoptosis, reduced endothelial cell activation or any combination thereof in comparison to an organ not treated with the composition. In preferred embodiments, administration of the composition to the organ results in the organ having at least 1%, or at least 2%, or at least 5%, or at least 10%, or at least 20%, or at least 50%, or at least 100% reduction in circulating MMP levels in comparison to a corresponding organ not treated with the composition. In preferred embodiments, administration of the composition to the organ results in the organ having at least 1%, or at least 2%, or at least 5%, or at least 10%, or at least 20%, or at least 50%, or at least 100% reduction in circulating markers of apoptosis in comparison to a corresponding organ not treated with the composition. In preferred embodiments, administration of the composition to the organ results in the organ having at least 1%, or at least 2%, or at least 5%, or at least 10%, or at least 20%, or at least 50%, or at least 100% reduction in endothelial cell activation in comparison to a corresponding organ not treated with the composition.

Some non-limiting examples of biological pathways that may be altered after administration of the composition include, but are not limited to, upregulation of the proteins implicated in (a) the extrinsic pathway, (b) transport of gamma-carboxylated protein precursors from the endoplasmic reticulum to the Golgi apparatus, (c) gamma-carboxylation of protein precursors, (d) removal of amino terminal pro-peptides from gamma-carboxylated proteins, and (e) gamma-carboxylation, transport, and amino-terminal cleavage of proteins. Some non-limiting examples of biological pathways that may be altered after administration of the composition include, but are not limited to, downregulation of proteins implicated in (a) the immune system, (b) the adaptive immune system, (c) cell surface interactions at the vascular wall, (d) co-stimulation by the CD28 family, and (e) hemostasis. Some non-limiting examples of molecular functions that may be altered after administration of the composition include, but are not limited to, (a) extracellular matrix binding, (b) receptor activity, (c) immunoglobulin receptor activity, (d) transmembrane receptor protein tyrosine kinase activity, and (e) peptidase activity. Some non-limiting examples of molecular functions that may be altered after administration of the composition include, but are not limited to, downregulating receptor activity.

Non-limiting examples of improved organ function after administration of the composition, when the organ may be, but is not limited to, a lung, may be, but are not limited to, increased dynamic compliance, increased gas exchange, decreased pulmonary vascular resistance, decreased wet/dry ratio, decreased Steen consumption per hour, decreased weight of the lung, and decreased observation of infiltrates on x-ray in comparison to a lung not treated with the composition.

In preferred embodiments, administration of the composition to the organ results in the organ having at least 1%, or at least 2%, or at least 5%, or at least 10%, or at least 20%, or at least 50%, or at least 100% increase in dynamic compliance in comparison to cells of a corresponding organ not treated with the composition. In preferred embodiments, administration of the composition to the organ results in the organ having at least 1%, or at least 2%, or at least 5%, or at least 10%, or at least 20%, or at least 50%, or at least 100% increase in gas exchange in comparison to cells of a corresponding organ not treated with the composition. In preferred embodiments, administration of the composition to the organ results in the organ having at least 1%, or at least 2%, or at least 5%, or at least 10%, or at least 20%, or at least 50%, or at least 100% decrease in pulmonary vascular resistance in comparison to cells of a corresponding organ not treated with the composition. In preferred embodiments, administration of the composition to the organ results in the organ having at least 1%, or at least 2%, or at least 5%, or at least 10%, or at least 20%, or at least 50%, or at least 100% decrease in wet/dry ratio in comparison to cells of a corresponding organ not treated with the composition. In preferred embodiments, administration of the composition to the organ results in the organ having at least 1%, or at least 2%, or at least 5%, or at least 10%, or at least 20%, or at least 50%, or at least 100% decrease in Steen consumption per hour in comparison to cells of a corresponding organ not treated with the composition. In preferred embodiments, administration of the composition to the organ results in the organ having at least 1%, or at least 2%, or at least 5%, or at least 10%, or at least 20%, or at least 50%, or at least 100% decrease in decrease in weight in comparison to cells of a corresponding organ not treated with the composition. In preferred embodiments, administration of the composition to the organ results in the organ having at least 1%, or at least 2%, or at least 5%, or at least 10%, or at least 20%, or at least 50%, or at least 100% decrease in observed infiltrates in comparison to cells of a corresponding organ not treated with the composition.

### III. Method of Improving Organ Transplantation

Disclosed herein is a method of improving organ transplantation, the method comprising administering the composition described above to an organ intended for transplantation. In some embodiments, the organ is, but is not limited to, a lung, a liver, or a kidney. In some embodiments, the organ is a xenotransplant organ. In a preferred embodiment, the lung is a human lung. In a preferred embodiment, the liver is a human liver. In a preferred embodiment, the kidney is a human kidney. In some embodiments, the organ treated with the composition is, but is not limited to, a bioengineered organ. The bioengineered organ may be a mechanical bioengineered organ, a biomechanical bioengineered organ, or a biological or bioartificial bioengineered organ. In some further embodiments, the bioengineered organ is, but is not limited to, a bioengineered lung, a bioengineered liver, or a bioengineered kidney.

In some embodiments, the method comprising administering a composition to treating an organ before or during ex vivo organ perfusion to improve organ transplantation, including at least one glycosaminoglycan component. In some embodiments, the composition is administered during ex vivo organ perfusion. In some embodiments, the composition contains at least one glycosaminoglycan component that includes but is not limited to high molecular weight hyaluronic acid, sulodexide, or both high molecular weight hyaluronic acid and sulodexide. High molecular weight hyaluronic acid was selected as it may be capable of inhibiting leakiness and restoring the glycocalyx.

In some embodiments, the method includes administering the composition to the organ before or during ex vivo organ perfusion. In some embodiments, the method includes administering the composition at the time of or after organ procurement. In some embodiments, the composition is administered before shipment. In some embodiments, the composition is administered after shipment. In some embodiments, the method includes administering the composition before ex vivo organ perfusion begins. In some embodiments, the method includes administering the composition during ex vivo organ perfusion. In some embodiments, the method includes administering the composition after ex vivo organ perfusion ends. In some embodiments, the method includes administering the composition before transplantation occurs. In some embodiments, the method includes administering the composition during transplantation. In some embodiments, the method includes administering the composition post-transplant. In embodiments wherein the organ is a bioengineered organ, the composition may be administrated before cell seeding or after cell seeding or at any of the times specified above. In some embodiments, the method includes, but is not limited to, administering the composition via perfusion. In some embodiments, the method includes administering the composition via perfusion for at least one hour. The method may include, but is not limited to, administering the composition via perfusion for at least 1, 2, 3, 4, 5, 6, 7, 8, 9, 10, 11, 12 or more hours. In some embodiments, the method may include administering the composition in doses over the course of perfusion. In some embodiments, the method includes, but is not limited to, administering the composition via injection. In some embodiments, the composition is administered to the organ via delivery to the airway.

In some embodiments, the method includes administering high molecular weight hyaluronic acid in a dose of about 0.01 mg to about 15 mg. In some embodiments, the method includes administering an initial dose of high molecular weight hyaluronic at hour 1 of ex vivo organ perfusion and optionally additional doses every 6 hours following the initial dose. In some embodiments, the method includes administering sulodexide in a dose of about 0.067 mg/kg to about 30 mg/kg. In some embodiments, the method includes administering an initial dose of sulodexide at hour 1 of ex vivo organ perfusion and optionally additional hourly following the initial dose. In some embodiments, the method includes administering an initial dose of sulodexide at hour 2 of ex vivo organ perfusion and optionally additional hourly following the initial dose. In some embodiments, the initial dose of high molecular weight hyaluronic acid and/or sulodexide are the same dose as the optional additional doses. In some embodiments, the initial dose of high molecular weight hyaluronic acid and/or sulodexide are different from the optional additional doses. In some embodiments, the method includes use of a solution for ex vivo organ perfusion including, but not limited to, dextran 40, sodium chloride, dextrose monohydrate, potassium chloride, calcium chloride dihydrate, sodium dihydrogen, phosphate dihydrate, sodium bicarbonate, magnesium chloride, hexahydrate, and human serum albumin (25%). In some embodiments, the method includes use of a solution for ex vivo organ perfusion that is, but is not limited to, Steen solution.

In some embodiments, the composition may include an antioxidant. The included antioxidant may be but is not limited to N-acetyl cysteine.

In some embodiments, the composition may also include a thrombolytic agent. The included thrombolytic agent may be but is not limited to human tissue type plasminogen activator.

In some embodiments, the composition may also include isolated mitochondria. In some further embodiments, the isolated mitochondria may be fresh mitochondria or frozen mitochondria. Isolated mitochondria may be used to improve the metabolism of the organ during EVLP.

In some embodiments, the composition may also include total parenteral nutrition (TPN). TPN may also be used to improve metabolism of the organ during EVLP.

In preferred embodiments, the method results in the organ treated with the composition exhibiting increased organ function. Examples of the improved organ function may be, but are not limited to, indicators associate with a reduction in edema or vascular leak.

Some non-limiting examples of improved organ function after treatment with the composition include reduced circulating MMP, reduced markers of apoptosis, reduced endothelial cell activation or any combination thereof in comparison to an organ not treated with the composition. In preferred embodiments, the organ treated with the composition has at least 1%, or at least 2%, or at least 5%, or at least 10%, or at least 20%, or at least 50%, or at least 100% reduction in circulating MMP levels in comparison to a corresponding organ not treated with the composition. In preferred embodiments, the organ treated with the composition has at least 1%, or at least 2%, or at least 5%, or at least 10%, or at least 20%, or at least 50%, or at least 100% reduction in circulating markers of apoptosis in comparison to a corresponding organ not treated with the composition. In preferred embodiments, the organ treated with the composition has at least 1%, or at least 2%, or at least 5%, or at least 10%, or at least 20%, or at least 50%, or at least 100% reduction in endothelial cell activation in comparison to a corresponding organ not treated with the composition.

Non-limiting examples of improved organ function after treatment with the composition, when the organ treated is a lung, may be, but are not limited to, increased dynamic compliance, increased gas exchange, decreased pulmonary vascular resistance, decreased wet/dry ratio, decreased Steen consumption per hour, decreased weight of the lung, and decreased observation of infiltrates on x-ray in comparison to a lung not treated with the composition. In preferred embodiments, the organ treated with the composition has at least 1%, or at least 2%, or at least 5%, or at least 10%, or at least 20%, or at least 50%, or at least 100% increase in dynamic compliance in comparison to cells of a corresponding organ not treated with the composition. In preferred embodiments, the organ treated with the composition has at least 1%, or at least 2%, or at least 5%, or at least 10%, or at least 20%, or at least 50%, or at least 100% increase in gas exchange in comparison to cells of a corresponding organ not treated with the composition. In preferred embodiments, the organ treated with the composition has at least 1%, or at least 2%, or at least 5%, or at least 10%, or at least 20%, or at least 50%, or at least 100% decrease in pulmonary vascular resistance in comparison to cells of a corresponding organ not treated with the composition. In preferred embodiments, the organ treated with the composition has at least 1%, or at least 2%, or at least 5%, or at least 10%, or at least 20%, or at least 50%, or at least 100% decrease in wet/dry ratio in comparison to cells of a corresponding organ not treated with the composition. In preferred embodiments, the organ treated with the composition has at least 1%, or at least 2%, or at least 5%, or at least 10%, or at least 20%, or at least 50%, or at least 100% decrease in Steen consumption per hour in comparison to cells of a corresponding organ not treated with the composition. In preferred embodiments, the organ treated with the composition has at least 1%, or at least 2%, or at least 5%, or at least 10%, or at least 20%, or at least 50%, or at least 100% decrease in weight in comparison to organ of a corresponding lung not treated with the composition. In preferred embodiments, the organ treated with the composition has at least 1%, or at least 2%, or at least 5%, or at least 10%, or at least 20%, or at least 50%, or at least 100% decrease in observed infiltrates in comparison to cells of a corresponding organ not treated with the composition.

Some non-limiting examples of biological pathways that may be altered after administration of the composition include, but are not limited to, upregulation of the proteins implicated in (a) the extrinsic pathway, (b) transport of gamma-carboxylated protein precursors from the endoplasmic reticulum to the Golgi apparatus, (c) gamma-carboxylation of protein precursors, (d) removal of amino terminal pro-peptides from gamma-carboxylated proteins, and (e) gamma-carboxylation, transport, and amino-terminal cleavage of proteins. Some non-limiting examples of biological pathways that may be altered after administration of the composition include, but are not limited to, downregulation of proteins implicated in (a) the immune system, (b) the adaptive immune system, (c) cell surface interactions at the vascular wall, (d) co-stimulation by the CD28 family, and (e) hemostasis. Some non-limiting examples of molecular functions that may be altered after administration of the composition include, but are not limited to, (a) extracellular matrix binding, (b) receptor activity, (c) immunoglobulin receptor activity, (d) transmembrane receptor protein tyrosine kinase activity, and (e) peptidase activity. Some non-limiting examples of molecular functions that may be altered after administration of the composition include, but are not limited to, downregulating receptor activity.

### EXAMPLES

The present technology is further illustrated by the following Examples, which should not be construed as limiting in any way. It is understood that the examples and embodiments disclosed herein are for illustrative purposes only and that various modifications or changes in light thereof will be suggested to persons skilled in the art and are to be included within the spirit and purview of this application.

### Example 1

To test whether circulating glycosaminoglycan components can affect glycocalyx integrity, two glycosaminoglycan components, high molecular weight hyaluronic acid (HMWHA) and sulodexide, were injected into marginal human lungs not suitable for transplantation undergoing 12 hours of EVLP. Four lungs were provided with the glycosaminoglycan components; eleven lungs were untreated controls. Lung function and circulating proteins were evaluated throughout EVLP.

### Methods

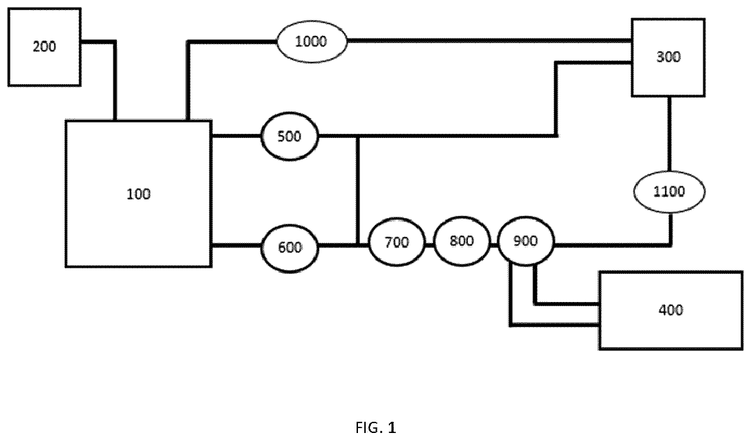

The EVLP setup used for the experiments discussed below consisted of equipment used in Toronto-style EVLP (FIG. 1). An organ chamber 100 may be connected to a ventilator 200 via a ventilator circuit. The organ chamber 100 may also be connected to a reservoir 300 via a leak return with a peristaltic pump 1000 disposed along the leak return 300. The reservoir 300 may be fluidly connected back to the organ chamber 100 via a PV/LA outflow only when human lungs are used in the EVLP with a pressure transducer 500 disposed along the PV/LA outflow. The reservoir 300 may also be connected back to the organ chamber via a PA inflow with a PA pump 1100, an oxygenator 900, a leukocyte filter 800, a flow sensor 700, and a pressure transducer 600 disposed along the PA inflow. The oxygenator 900 may also be connected to a heater/chiller unit 400 via a water inflow and a water outflow. The PV/LA outflow and the PA inflow may be fluidly connected by a leak return.

For the following experiments, the organ chamber, XVIVO 19020, was connected to a perfusion set and primed with STEEN solution followed by addition of imipenem and heparin. The organ chamber connected the lung to a perfusion set. Perfusate flowed from an external reservoir to a centrifugal pump head, through an oxygenator/heat exchanger, then a leukocyte filter before entering the pulmonary artery. Left atrial outflow was returned from the cannula directly to the reservoir. Perfusate was returned to the reservoir using a roller pump.

After perfusion was started, the flow rate was slowly increased to 40% of target cardiac output based on the donor ideal bodyweight. Volume-controlled ventilation was used with a tidal volume of 7 mL/kg ideal body weight, 5 cm H₂O PEEP, 1:2 I: E ratio, and a respiration rate of 7 breaths/minute, FiO₂ 0.21. Bronchoscopy and recruitment maneuvers were performed up to a maximum peak airway pressure of 25 cm H₂O. Blood gas assessments were completed at 1 hour of perfusion and hourly thereafter at tidal volume 10 mL/kg ideal body weight, FiO₂ of 1.0, and respiration rate of 10 breaths/minute. Treatment with the composition comprising glycosaminoglycans consisted of 2 mg hyaluronic acid (HA) added into the EVLP circulate at hour 1 and re-dosed every 6 hours. 500 μg sulodexide was added at hour 2 in a single dose.

TABLE 1
Human lung donor demographics

| EVLP | | | | | | |
|---|---|---|---|---|---|---|
| Duration | | | | Height | Weight | CIT |
| (hours) | Treatment | Sex | Age | (cm) | (kg) | HH:MM |
| 12 | GAG | M | 59 | 173 | 88 | 21:13 |
| | treatment | | | | | |
| 12 | GAG | F | 58 | 170 | 64 | 17:17 |
| | treatment | | | | | |
| 12 | GAG | M | 59 | 160 | 103 | 17:02 |
| | treatment | | | | | |
| 24 | Control | M | 49 | 175 | 68 | 07:22 |
| 24 | Control | M | 65 | 178 | 80 | 21:31 |
| 24 | Control | F | 54 | 165 | 86 | 20:17 |
| 3 | Control | F | 29 | 155 | 67 | 17:23 |
| 24 | Control | M | 62 | 178 | 85 | 08:25 |
| 4 | Control | M | 33 | 175 | 82 | 20:38 |
| 24 | Control | M | 71 | 177 | 83 | 20:49 |
| 6 | Control | M | 61 | 165 | 89 | 05:36 |
| 10 | Control | F | 38 | 153 | 94 | 20:25 |
| 24 | Control | F | 72 | 165 | 100 | 11:19 |
| 12 | Control | F | 36 | 170 | 79 | 18:28 |
| 2 | Control | F | 43 | 165 | 65 | 17:49 |

M: male,
F: female.
CIT: cold ischemia time,
CIT HH:MM: cold ischemia time hour:minute

### Results

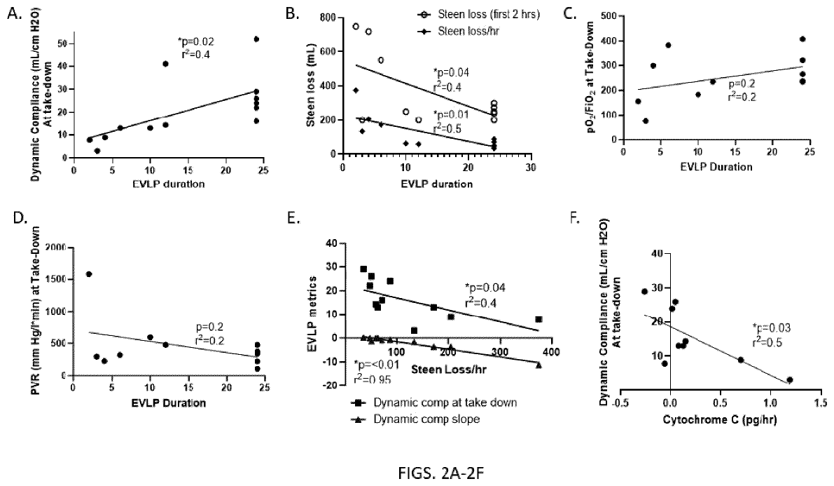

Lung function was measured by a variety of assays initially to determine changes during EVLP without treatment. First, dynamic compliance was measured at take-down, or at the end of EVLP, for untreated lungs for EVLP duration permitted (FIG. 2A). Dynamic compliance measures the lung's elasticity, which is a read-out of the organ's health. It was found that dynamic compliance is positively correlated with EVLP duration, indicating that increased dynamic compliance of the lungs allowed for longer EVLP.

Second, loss of Steen solution, first, for total loss in the first 2 hours of EVLP and, second, per hour of EVLP were plotted against EVLP duration (FIG. 2B). Steen loss is a readout of loss of vascular integrity and leakage of liquids from the vasculature into the parenchyma of the lung. A correlation between high Steen loss in the first two hours of EVLP or per hour of EVLP and low duration of EVLP was observed, indicating that Steen loss is indicative of reduced EVLP survivability.

Third, the ratio of partial pressure of oxygen in arterial blood (pO₂) to the fraction of inspired oxygen (FiO₂) measured at take-down was plotted against EVLP duration (FIG. 2C). This ratio can be used to characterize lung dysfunction as severe PGD is characterized by a ratio of less than 200 mmHg. A weak correlation between higher pO₂/FiO₂ and decreased EVLP duration was observed.

Fourth, pulmonary vascular resistance (PVR) was measured at take-down and plotted against the duration of EVLP (FIG. 2D). PVR indicates the pressure in the arteries that supply blood to the lungs. Increased PVR is associated with remodeling in response to chronic pulmonary vascular injury. A weak correlation between increased PVR and decreased EVLP duration was observed.

Fifth, dynamic compliance at take-down and average dynamic compliance were plotted against Steen loss per hour (FIG. 2E). Measurement of dynamic compliance at take-down and average dynamic compliance represents an effective way to capture a metric that is increasing or decreasing over time and may have very different starting values. An inverse relationship was seen between both dynamic compliance measurements and Steen loss such that high dynamic compliance either at take-down or on average is correlated with lower levels of Steen loss per hour during EVLP.

Sixth, dynamic compliance at take-down was plotted against the rate of accumulation of cytochrome C, a protein found in mitochondria and released during apoptosis (FIG. 2F). The rates of accumulation for the proteins were the value for the slope of levels of circulating proteins measured during EVLP. Increased rates of accumulation of circulating cytochrome C were associated with cell death. Dynamic compliance is negatively correlated with circulating cytochrome C levels, suggesting that high levels of dynamic compliance indicate reduced cell death.

Seventh, dynamic compliance at take-down was plotted against the rate of accumulation of syndecan-1, a transmembrane heparin sulfate proteoglycan that is associated with endothelial cell activation, damage to the glycocalyx, and deteriorated lung quality (FIG. 2G). The accumulation of circulating syndecan-1 over the course of EVLP was represented as the slope of each protein. High levels of syndecan-1 were associated with decreased dynamic compliance.

Eighth, dynamic compliance at take-down was plotted against the rate of accumulation of two additional perfusate indicators, epithelial cell adhesion molecule (EpCAM) and vascular cell adhesion molecule 1 (VCAM-1) (FIG. 2H). EpCAM is a transmembrane glycoprotein that mediates cell adhesion in the epithelium, and circulating EpCAM indicates a loss of epithelial integrity. VCAM-1 is an adhesion molecule expressed in endothelial cells in response to cellular stress, such as cytokines. High levels of circulating VCAM-1 are indicative of endothelial cell activation, glycocalyx damage, and are associated with PGD. Much like Cytochrome C and syndecan-1, the rates of accumulation of both EpCAM and VCAM-1 were negatively correlated with dynamic compliance, indicating that increased levels of negative markers of lung function and health are associated with decreased dynamic compliance.

Finally, dynamic compliance at take-down was plotted against the rates of accumulation of circulating IL-10 levels (FIG. 2I). IL-10 is a cytokine that is associated with the inflammation and the immune response. Much like the other perfusate indicators, higher rates of accumulation of IL-10 levels were associated with lower dynamic compliance.

Taken together, these results suggest that breakdown of the glycocalyx and thus the vascular barrier, as seen by measuring Steen loss and the secretion of key proteins, are associated with truncated EVLP duration and poor lung function. These data also suggest that barrier function is a large driving factor for poor performance as evidenced by the relationships observed between lung function and proteins associated with the glycocalyx degradation.

It was also found that proteins detected via bronchoalveolar lavage (BAL) at hour 1 of EVLP may be associated with lung function. Two such sets of proteins are matrix metalloproteinases (MMPs) and tissue inhibitors of metalloproteinases (TIMPs). The levels of MMPs and the ratio of MMPs to TIMPs, both measured at hour 1 of EVLP, were plotted against several markers of lung function, including Steen loss per hour (FIG. 3A), dynamic compliance over time during EVLP (FIG. 3B), and dynamic compliance at take-down (FIG. 3C). Consistently for all samples, high levels of MMP initially detected at hour 1 was negatively correlated with Steen loss per hour, as all sample with initial high levels of MMP exhibited high rates of Steen loss. This same trend was observed as well for the MMP/TIMP ratio, since high ratios of MMP/TIMP at hour 1 of EVLP were correlated with high rates of Steen loss per hour.

The same protein measurements were analyzed against dynamic compliance over the course of EVLP. High levels of MMP detected at hour 1 of EVLP did show a correlation with decreased compliance over the course of EVLP, and a similar correlation was seen for the ratio of MMPs to TIMPs.

Lastly, the initial MMP levels and ratio of MMPs to TIMPs were compared to dynamic compliance specifically at take-down. Much like MMP levels for average dynamic compliance, there is an inverse correlation between the MMP levels and dynamic compliance at take-down, with high levels of MMPs associated with reduced dynamic compliance. The same trend is observed for the MMP/TIMP ratio, where a high MMP/TIMP ratio is correlated with decreased dynamic compliance at take-down.

EpCAM levels were also measured at hour 1 of EVLP and plotted against dynamic compliance at take-down (FIG. 3D). Increased EpCAM levels were associated with decreased dynamic compliance at takedown. Additionally, high EpCAM levels were also correlated with high Steen loss per hour. These data together indicate that EpCAM may also be used as a predictor of lung function over time.

In sum, initial BAL protein levels, including MMPs and TIMPs, could predict lung function over the duration of EVLP and may act as early indicators of poor lung function at later time points.

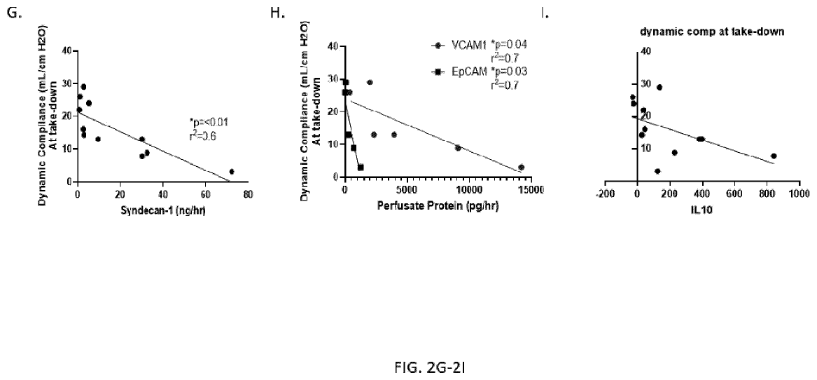

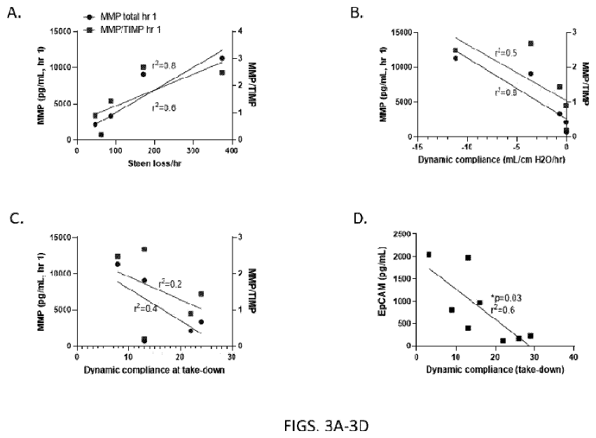

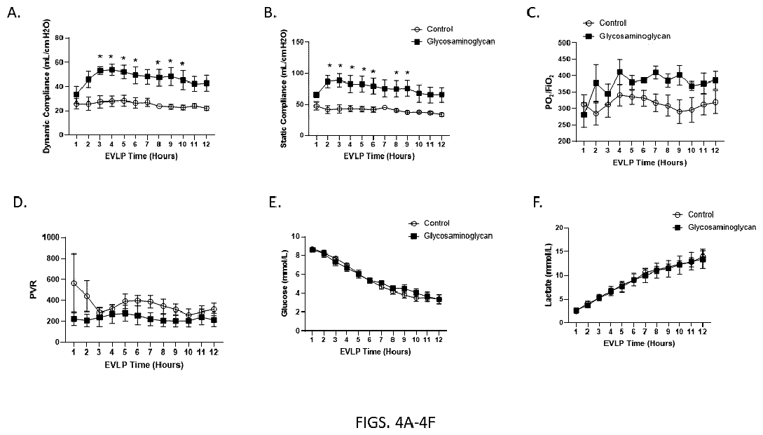

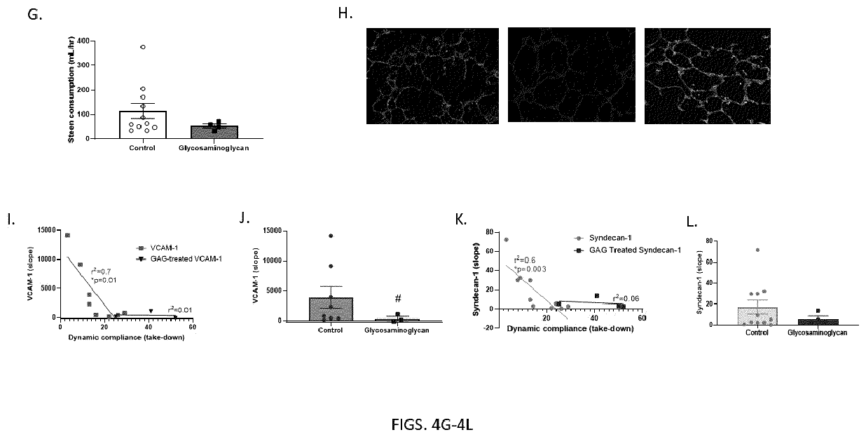

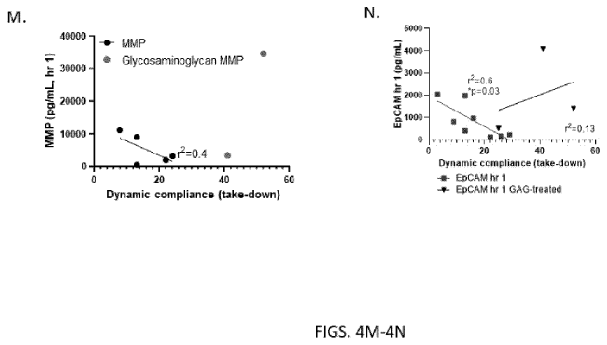

In order to determine if treatment with the composition comprising glycosaminoglycans affected lung function during EVLP, similar assays were conducted with treated and untreated lungs. First, dynamic compliance was measured for untreated and treated lungs each hour during EVLP. The lungs treated with the composition comprising the glycosaminoglycan components consistently exhibits higher dynamic compliance, indicating that the organs are healthier than the untreated samples (FIG. 4A). In addition to measuring dynamic compliance, static compliance, which measures the elastic properties of the lungs when there is no airflow, such as during an inspiratory pause, also reflected the same trend observed for dynamic compliance, with the treated samples exhibiting higher static compliance over the course of EVLP as compared to the control lungs (FIG. 4B)

Next, the ratio of the partial pressure of oxygen in arterial blood (PO₂) to the fraction of inspired oxygen (FiO₂) was measured for treated and untreated lungs. Over the 12-hour EVLP, the treated lungs consistently exhibited a higher ratio than the untreated lungs, suggesting that treatment with the combination of high molecular weight hyaluronic acid and sulodexide are sufficient to mitigate one of the markers of PGD (FIG. 4C).

Fourth, pulmonary vascular resistance (PVR) was measured for the duration of EVLP. PVR indicates the pressure in the arteries that supply blood to the lungs, and increased PVR is associated with remodeling in response to chromic pulmonary vascular injury. The treated lungs consistently exhibited lower PVR than untreated lungs for each time point measured, indicating treatment may prevent or decrease injury or inflammation following injury (FIG. 4D).

Fifth, glucose concentration was measured for the duration of EVLP for both treated and untreated lungs. Higher consumption of glucose is associated with worse lung function. Both the treated and the untreated lungs exhibited similar glucose concentrations for the duration of EVLP, suggesting that glycocalyx-associated biomolecules have little effect on glucose consumption (FIG. 4E). Further, lactate production was also measured for the duration of EVLP for treated and control lungs (FIG. 4F). Lactate production is associated with worse lung function, and, much like glucose consumption, there was no effect on lactate production when the lungs were treated with the composition comprising glycosaminoglycans.

Steen solution consumption per hour was calculated for the treated and untreated lungs. Steen solution consumption, much like Steen loss, is indicative of vascular integrity and leakage of liquids from the vasculature into the parenchyma of the lung. This assay measures the volume of Steen solution taken up by the lung from the perfusate during EVLP, and consistent uptake results in edema and decreased lung function. The untreated lungs exhibited an average Steen solution consumption per hour of about 100 mL/hr, while the treated lungs had an average Steen solution consumption per hour of about 50 mL/hr (FIG. 4G).

Next, samples of lung tissue were stained for hyaluronic acid (HA) presence (FIG. 4H). Hyaluronic acid is a glycosaminoglycan component that is implicated in glycocalyx integrity. While hyaluronic acid is clearly present after three hours of EVLP, untreated lungs show a dramatic decrease in hyaluronic acid. However, lungs that have been treated with the composition comprising glycosaminoglycans show robust hyaluronic acid presence even after 24 hours of EVLP. Thus, treatment with the composition comprising glycosaminoglycans increased glycocalyx presence at later EVLP timepoints.

Circulating VCAM-1 levels were measured in the treated and untreated lungs and plotted against dynamic compliance at take-down to determine if treatment with the composition comprising glycosaminoglycans was sufficient to alter accumulation of VCAM-1 levels or to alter the correlation between VCAM-1 and dynamic compliance at take-down (FIGS. 4I and 4J). The results indicate that the samples treated with the composition comprising glycosaminoglycans exhibited higher dynamic compliance than the untreated lungs, including those that had lower VCAM-1 levels than the treated lungs, suggesting that the GAG treatment prevents the degradation of the glycocalyx over time in EVLP.

Treatment with composition comprising glycosaminoglycans was also able to prevent loss of lung function predicted by syndecan-1, as dynamic compliance at take-down for lungs treated with the composition comprising glycosaminoglycans was consistently higher than untreated lungs, including those with reduced levels of syndecan-1 (FIGS. 4K and 4L).

Administration of the composition comprising glycosaminoglycans was also sufficient to resolve early indicators of poor lung function. Both treated and untreated lungs were assayed for MMP levels and circulating EpCAM levels at hour 1 of EVLP and then dynamic compliance at take-down (FIGS. 4M and 4N). Based on the correlation identified between EpCAM and lung function, it would be predicted that the lungs would exhibit reduced dynamic compliance at take-down. However, the results indicate that the lungs treated with the glycosaminoglycan components exhibited higher dynamic compliance than untreated lungs that had lower initial EpCAM levels than the treated lungs. This finding further indicates that treatment with the glycosaminoglycan components can resolve early indicators of poor lung function, such as early loss of adhesion in the epithelia.

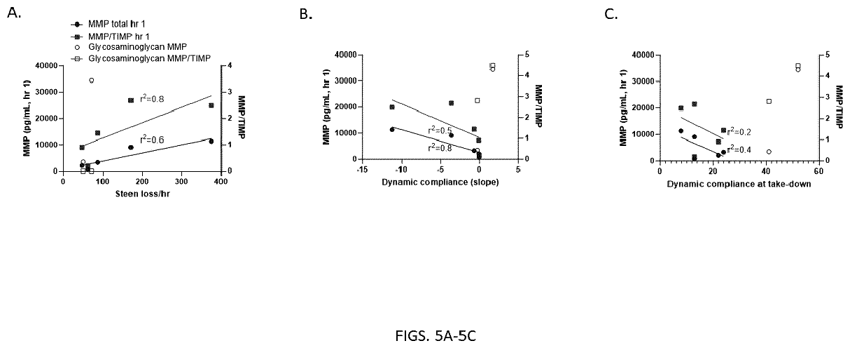

Some samples treated with the glycosaminoglycan components, depicted as the hollow circles and squares on FIGS. 5A-5C, exhibited high initial levels of MMP and high initial MMP/TIMP ratio, which would predict an increase in Steen loss per hour, a decrease in dynamic compliance overall, and a decrease in dynamic compliance at the end of EVLP. However, the treated lungs with the high initial levels exhibited similar Steen loss per hour and dynamic compliance for both time periods to treated lungs that did not have high initial levels of BAL proteins. Thus, treatment with the composition comprising the two glycosaminoglycan components was able to resolve the early indicators and promote better lung function over time.

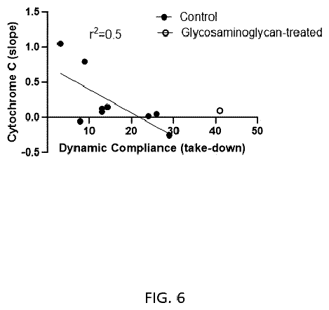

Accumulation of circulating cytochrome c protein over the course of EVLP for the treated and untreated lungs was plotted against dynamic compliance at take-down (FIG. 6). While the slope of the line depicts the observed correlation between cytochrome C levels and dynamic compliance, the treated lung sample does not fall along the slope, indicating that treatment with the composition comprising glycosaminoglycans is sufficient to alter the relationship between this marker as well and lung function,

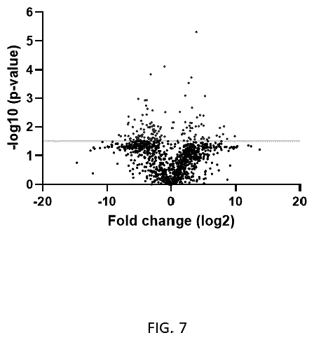

To understand what molecular pathways were affected by administering the composition comprising glycosaminoglycans, cytokine and MMP protein arrays were employed, and 1210 related proteins were evaluated. The rate of accumulation and fold change was determined for each circulating protein during EVLP for both control and treated lungs (FIG. 7). All proteins with a log 10 p-value of 1.5 and higher were entered into FunRich (Mathivanan lab) to evaluate key biological pathways and molecular functions upregulated and downregulated.

TABLE 2
Proteins Impacted by Treatment

| Protein | Fold Change (log2) | −log10 (P-Value) |
|---|---|---|
| LH | −1.03415 | 4.119873 |
| Numb | −3.18045 | 3.842675 |
| Kalllikrein 1 | −5.15319 | 2.982457 |
| GASP-1 | −3.81478 | 2.935242 |
| Neuroglycan C | −4.06808 | 2.930946 |
| Presenilin 1 | −3.8918 | 2.74092 |
| GLP-1 | −3.69924 | 2.634585 |
| SLAM | −2.9056 | 2.57863 |
| B7-2 | −0.90641 | 2.519559 |
| IP-10 | −3.82925 | 2.428145 |
| Sirtuin 5 | −4.46982 | 2.415449 |
| HIF-1 beta | −6.6448 | 2.363397 |
| Angiostatin | −5.69211 | 2.251188 |
| BTC | −4.6768 | 2.210115 |
| EDAR | −3.0432 | 2.151127 |
| IL-20 RA | −6.68491 | 2.141293 |
| E-Cadherin | −3.27012 | 2.113384 |
| Glypican 2 | −4.48795 | 2.100114 |
| Endoglycan | −6.74415 | 2.087858 |
| ULBP-3 | −3.81113 | 2.061536 |
| OMgp | −2.72501 | 2.058768 |
| CD177 | −2.73108 | 2.050982 |
| BLAME | −2.65447 | 2.032659 |
| Dtk | −2.97169 | 2.01999 |
| CA5A | −2.04309 | 2.016143 |
| CA2 | −3.29019 | 2.001215 |
| WIF-1 | 3.837292 | 5.304575 |
| OSM | 3.046234 | 3.731556 |
| VEGF-D | 2.633879 | 3.542668 |
| Legumain | 2.090577 | 3.095799 |
| Cadherin-13 | 5.189275 | 3.081263 |
| MMR | 3.169234 | 2.670925 |
| Cathepsin B | 1.262604 | 2.523261 |
| ENA-78 | 4.42015 | 2.413377 |
| Ephrin-A4 | 4.949368 | 2.391605 |
| ROR1 | 3.947322 | 2.334819 |
| Serpin B2 | 2.093928 | 2.165782 |
| TRAIL R3 | 0.301449 | 2.158901 |
| Chemerin | 2.903455 | 2.143197 |
| Podoplanin | 3.093802 | 2.138877 |
| KIR3DL1 | 4.627933 | 2.091785 |
| CD200 R1 | 4.966116 | 2.044824 |
| SPARCL1 | 2.775199 | 2.036741 |
| Nogo Receptor | 4.219458 | 2.022411 |
| Kell | 8.15586 | 2.018853 |
| FAP | 3.742256 | 2.018009 |

TABLE 3
Pathways Upregulated by Treatment

| | P-Value |
|---|---|
| | (hypergeometric test) |
| Upregulated Biological Pathway | |
| Extrinsic pathway | 1.82E−05 |
| Transport of gamma-carboxylated protein | 5.07E−05 |
| precursors from the endoplasmic reticulum | |
| to the Golgi apparatus | |
| Gamma-carboxylation of protein precursors | 6.52E−05 |
| Removal of amino terminal pro-peptides | 8.14E−05 |
| from gamma-carboxylated proteins | |
| Gamma-carboxylation, transport, and | 0.000119 |
| amino-terminal cleavage of proteins | |
| Upregulated Molecular Function | |
| Extracellular matrix binding | 0.001324 |
| Receptor activity | 0.001508 |
| Immunoglobulin receptor activity | 0.001985 |
| Transmembrane receptor protein tyrosine | 0.036463 |
| kinase activity | |
| Peptidase activity | 0.037743 |

TABLE 4
Pathways Downregulated by Treatment

| | P-Value |
|---|---|
| | (hypergeometric test) |
| Downregulated Biological Pathway | |
| Immune system | 1.51E−05 |
| Adaptive immune system | 1.57E−05 |
| Cell surface interactions at the vascular wall | 2.59E−05 |
| Costimulation by the CD28 family | 0.000277 |
| Hemostasis | 0.001852 |
| Downregulated Molecular Function | |
| Receptor activity | 7.7E−06 |

Taken together, the studies identified a series of markers and indicators of negative outcomes after lung transplantation. Generally, EVLP function and duration were associated with glycocalyx integrity. Treatment with a composition of glycosaminoglycan components high molecular weight hyaluronic acid and sulodexide resulted in increased lung function and resolved early indicators associated with negative outcomes and displayed increased dynamic compliance trends as compared to untreated controls. The treatment also resolved early indicators of barrier disruption in the perfusate and BAL proteins and reduced circulating and tissue markers of endothelial damage and tissue edema. In all, these results suggest that administration of the composition comprising glycosaminoglycans leads to repair or enhancement of the endothelial glycocalyx during EVLP. Thus, the composition may act to prevent damage to organs and negative transplantation outcomes.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions or biological systems, which can, of course, vary. It is also to be understood that the terminology used herein is for the purpose of describing particular embodiments only and is not intended to be limiting.

All patents, patent applications, provisional applications, and publications referred to or cited herein are incorporated by reference in their entirety, including all figures and tables, to the extent that are not inconsistent with the explicit teachings of this specification.

What is claimed is:

1. A composition for treating an organ, comprising an effective amount of at least one glycosaminoglycan component.

2. The composition of claim 1, wherein the at least one glycosaminoglycan component comprises high molecular weight hyaluronic acid, sulodexide, or a combination of high molecular weight hyaluronic acid and sulodexide.

3. The composition of claim 2, further comprising at least one additional component selected from the group consisting of: isolated mitochondria, total parenteral nutrition (TPN), an antioxidant and a thrombolytic agent.

4. The composition of claim 3, wherein the antioxidant is N-acetyl cysteine.

5. The composition of claim 3, wherein the thrombolytic agent is human tissue type plasminogen activator.

6. A method of treating an organ, comprising contacting the organ with a composition of claim 1.

7. The method of claim 6, wherein the glycosaminoglycan components comprise high molecular weight hyaluronic acid, sulodexide, or a combination of high molecular weight hyaluronic acid and sulodexide.

8. The method of claim 7, wherein high molecular weight hyaluronic acid is present in an amount of 0.01 mg to 15 mg.

9. The method of claim 7, wherein sulodexide is present in an amount of 0.067 mg/kg to 30 mg/kg.

10. The method of claim 7, wherein the composition further comprises isolated mitochondria, total parenteral nutrition (TPN), an antioxidant and/or a thrombolytic agent.

11. The method of claim 6, wherein contacting the organ comprises perfusing the organ with the composition.

12. The method of claim 11, wherein the organ is perfused with the composition for at least one hour.

13. The method of claim 6, wherein contacting the organ comprises administration of one or more doses of the composition to the organ before transplantation.

14. The method of claim 13, wherein contacting the organ occurs at a time selected from the group consisting of: (a) after procurement of the organ from a donor, (b) before shipment of the organ, (c) before EVLP, (d) during EVLP, (e) after EVLP, and (f) immediately before transplant.

15. The method of claim 6, wherein the organ exhibits a decrease in circulating MMP, markers of apoptosis, and/or endothelial cell activation compared to an organ perfused with a solution lacking glycosaminoglycan components.

16. The method of claim 6, wherein the organ is a lung, a liver, or a kidney.

17. The method of claim 6, wherein the organ is a bioengineered organ.

18. The method of claim 6, wherein an initial dose of high molecular weight hyaluronic acid is administered at hour 1 of ex vivo organ perfusion and optionally additional doses administered every 6 hours following the initial dose.

19. The method of claim 7, wherein an initial dose of sulodexide is administered at either hour 1 or hour 2 of ex vivo organ perfusion, and optionally additional doses administered hourly following the initial dose.

20. The method of claim 6, wherein the contacting the organ comprises delivering the composition to the airway of the organ.
